(19) United States
(12) Patent Application Publication
(10) Pub. No.: US 2019/0307908 A1
(43) Pub. Date: Oct. 10, 2019

(54) NANOEMULSION AGENTS FOR ULTRASOUND DIAGNOSTIC AND THERAPY

(71) Applicant: University of Washington, Seattle, WA (US)

(72) Inventors: Lilo D. Pozzo, Seattle, WA (US); David Li, Seattle, WA (US); Yi-Ting Lee, Seattle, WA (US); Matthew O'Donnell, Seattle, WA (US)

(21) Appl. No.: 16/374,502

(22) Filed: Apr. 3, 2019

Related U.S. Application Data
(60) Provisional application No. 62/653,345, filed on Apr. 5, 2018.

(51) Int. Cl. A61K 49/22; A61K 9/107; A61K 41/00; A61B 8/08; A61N 7/00

(52) U.S. Cl. CPC A61K 49/223; A61K 9/1075; A61N 2007/0039; A61B 8/481; A61N 7/00; A61K 41/0028

(57) ABSTRACT

The present disclosure features, a kit, including a first compartment including a volatile fluorinated compound dissolved in a C₁₋₆ alcohol; and a second compartment including water. When the contents of the first and second compartments are mixed, spontaneous nucleation of nanodroplets of the volatile fluorinated compound can form in the aqueous phase to provide a dispersion of nanodroplets in the aqueous phase. The nanodroplets can be used to generate nanobubbles or microbubbles in ultrasound imaging.

### CROSS-REFERENCE(S) TO RELATED APPLICATION(S)

This application claims the benefit of U.S. Patent Application No. 62/653,345, filed Apr. 5, 2018, the disclosure of which is hereby incorporated by reference in its entirety.

### STATEMENT OF GOVERNMENT LICENSE RIGHTS

This invention was made with government support under Grant No. R01 HL125339, awarded by the National Institutes of Health. The government has certain rights in the invention.

### BACKGROUND

Contrast-enhanced imaging with exogenous contrast agents is a rapidly developing technique for both photoacoustic (PA) and ultrasound (US) systems. In particular, perfluorocarbon (PFC) gas-filled nanobubbles or microbubbles are a medically approved contrast agent for diagnostic ultrasound imaging. In addition to vascular and cardiac imaging, nanobubbles or microbubbles are also being tested for drug delivery and cavitation-based therapies. For example, PFC gas-filled nanobubbles or microbubbles have been demonstrated to be useful in therapeutic applications, such as tumor treatment, drug delivery, and gene therapy. They are typically constrained to endovascular applications because of their large size (>1 μm in diameter). Because of recent interest in nanobubbles or microbubbles for ultrasound-based theranostics (combining diagnostic imaging with therapy), great effort has been made in synthesizing nanobubbles or microbubbles small enough to diffuse between tight junctions and penetrate into diseased tissues. To freely diffuse past the vessel walls and into tissue, agents must be smaller than about 200 nm in diameter. Producing such agents has proven difficult because bubbles at those scales are unstable and have short lifetimes due to accelerated dissolution into the surrounding liquid due to a higher Laplace pressure. Moreover, several studies have concluded that as the bubble diameter decreases, the effective membrane stiffness increases. This in turn causes an increase in bubble resonant frequency and reduced echogenic properties.

Liquid PFC nanodroplets can serve as an alternative to gaseous nanobubbles or microbubbles for medical ultrasound applications. As liquid droplets, the emulsions can be stable for days or weeks. Moreover, prior to activation, they are transparent to ultrasound and provide virtually no ultrasound imaging contrast. However, upon applying a high amplitude acoustic pulse, droplets can be selectively vaporized in a region of interest to form bubbles up to five times the diameter of the initial droplet. After “activation,” gas nanobubbles or microbubbles can be used in the same manner as conventional microbubbles. In addition to contrast enhanced imaging, phase-change contrast agents have also been actively researched in therapeutic ultrasound applications including embolotherapy, histotripsy, drug delivery, and photoacoustic imaging. PFC droplets with diameters under 200 nm can diffuse out of blood vessels and into tissues for extravascular imaging and/or therapy. However, as the droplet diameter decreases, they also experience a stabilizing effect preventing spontaneous vaporization, and require an increase in the acoustic activation threshold.

There are two debated mechanisms for explaining the increased stability of dispersions of nanodroplets (also referred to herein as “nanoemulsions”): increased contributions from the Laplace pressure and homogeneous nucleation. The long-standing hypothesis has been that the increase in Laplace pressure with reducing droplet diameter results in an increase in internal pressure in the droplet and thus a suppression of boiling. However, without wishing to be bound by theory, it is believed that Laplace pressure does not sufficiently explain the enhanced droplet stability. Instead, homogeneous nucleation theory predicts a much greater energy barrier to droplet vaporization that better matches experimental measurements. Nevertheless, the Laplace pressure and homogeneous nucleation theory both suggest an increase in droplet stability with decreasing diameter, requiring an increase in acoustic pressure needed to initiate droplet vaporization, potentially to levels beyond FDA limits. The FDA limit is defined as the MI<1.9, where MI=P/\sqrt{f}, P is the peak negative pressure in MPa and f is the frequency in MHz. Although superharmonic focusing of the acoustic wave in droplets leads to a decrease in acoustic vaporization threshold with increasing frequency, which can reduce the activation threshold to within FDA limits, a reduction in focal gain is observed when the droplet is much smaller than an acoustic wavelength (e.g., nanodroplets). Low boiling point PFCs can help reduce the vaporization threshold to within FDA limits. Often, natively gaseous PFCs must be used to synthesize nanodroplets with a sufficiently low acoustic pressure threshold for clinical purposes.

It can be very challenging to maintain low boiling point PFCs, such as perfluorobutane (T_{Boiling} (i.e., boiling point at 1 atm pressure)=−2° C.) and perfluoropropane (T_{Boiling}=−37° C.), in their liquid phase during droplet synthesis using conventional methods such as sonication, high-speed shaking, and homogenization. For example, a high-speed shaker can be used to produce nanobubbles or microbubbles for ultrasound imaging, and sonication can be used to form liquid perfluorocarbon droplets. However, the quality of the bubbles a shaker produces is unsatisfactory both in the uniformity of the bubble size and the stability of the bubbles. In some cases, cryogenic conditions must be used during emulsification. Alternatively, condensing microbubbles to form nanodroplets is sometimes used to form nanodroplets from low boiling point PFCs. However, microbubble condensation is limited to gaseous PFCs.

There is presently a need for methods of making nanodroplets, nanobubbles, and microbubbles of volatile fluorinated compounds that are stable and have good echogenic properties relative to conventional microbubbles. The present disclosure seeks to fulfill these needs and provides further related advantages.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the present disclosure features a kit, including a first compartment including a volatile fluorinated compound dissolved in a C₁₋₆ alcohol; and a second compartment including water; wherein the volatile fluorinated compound is present in the first compartment at a concentration of 0.2% or more and 4% or less by volume; and the volume ratio of the volatile fluorinated compound and C₁₋₆ alcohol:water is 24:1 to 500:1.

In another aspect, the present disclosure features a nanodroplet composition, including a dispersion of a volatile fluorinated compound in the form of nanodroplets in an aqueous liquid, wherein the nanodroplets have an average volume of 1.1×10⁵ nm³ or more and 4.2×10⁹ nm³ or less; the nanodroplets are present in the aqueous liquid at a concentration ranging from 1×10⁷ to 1×10¹⁴ nanodroplets per ml of aqueous liquid. In yet another aspect, the present disclosure features a method for making the nanodroplet composition above, including providing a first solution including a volatile fluorinated compound dissolved in a C₁₋₆ alcohol and a second solution including water, and mixing the first and second solutions to provide the nanodroplet composition. In some embodiments, the first and second solutions do not include any of polyvinyl alcohol, polypyrrole, and pyrrole.

In yet another aspect, the present disclosure features a method of generating nanobubbles or microbubbles, including exposing the nanodroplet composition above to ultrasound to vaporize the volatile fluorinated compound-containing nanodroplets, thereby providing volatile fluorinated compound-containing nanobubbles or microbubbles having a diameter ranging from 120 nm to 10 μm.

In yet a further aspect, the present disclosure features a therapeutic or diagnostic method, including administering a nanodroplet composition above into a subject in need thereof, wherein the nanodroplet composition includes a dispersion of a volatile fluorinated compound in the form of nanodroplets in an aqueous liquid, wherein the nanodroplets have an average volume of 1.1×10⁵ nm³ or more and 4.2×10⁹ nm³ or less, and the nanodroplets are present in the aqueous solution at a concentration of 1×10⁷ to 1×10¹⁴ nanodroplets per ml of aqueous solution; and exposing the injected nanodroplet composition to ultrasound to vaporize the nanodroplets, thereby providing nanobubbles or microbubbles having a diameter from 120 nm to 10 p.m. In some embodiments, the nanodroplet composition does not include any of polyvinyl alcohol, polypyrrole, and pyrrole.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1 is a drawing of embodiments of the kits of the present disclosure.

FIG. 2 is a drawing of a method of the present disclosure for nanodroplet production by spontaneous nucleation of liquid nanodroplets in solution.

FIG. 3A is an ouzo phase diagram of perfluorohexane. The ouzo region (conditions leading to droplet formation) is identified with the filled area with a dotted line border. Droplets nucleate in a narrow region relative to the entire ternary diagram.

FIG. 3B is an ouzo phase diagram of perfluoropentane. The ouzo region (conditions leading to droplet formation) is identified with the filled area with a dotted line border. Droplets nucleate in a narrow region relative to the entire ternary diagram.

FIG. 3C is an ouzo phase diagram of perfluorobutane. The ouzo region (conditions leading to droplet formation) is identified with the filled area with a dotted line border. Droplets nucleate in a narrow region relative to the entire ternary diagram.

FIG. 3D is an ouzo phase diagram of perfluoropropane. The ouzo region (conditions leading to droplet formation) is identified with the filled area with a dotted line border. Droplets nucleate in a narrow region relative to the entire ternary diagram.

FIG. 4 is an illustration of an embodiment of the use of the kits of the present disclosure.

FIG. 5A is a graph of size distribution measured using dynamic light scattering of various PFCs prepared using the ouzo synthesis method.

FIG. 5B is a graph of the size evolution of droplets stored at room temperature or refrigerated over time.

FIG. 5C is a graph of the average size of perfluorohexane droplets synthesized as a function of ethanol and PFC saturation in ethanol prior to nucleation. The error bars represent one standard deviation of the size distribution.

FIG. 6A is a graph of cavitation thresholds of pure PFC droplets compared to water.

FIG. 6B is a graph of cavitation thresholds using perfluorocarbon mixtures. The inset figure in panel B shows the 50% cavitation threshold of the droplets as a function of perfluorohexane and perfluorobutane mixture ratios. The cavitation threshold of the PFC blended droplets fits the linear function of P_{Threshold}=P_{PFC}_{₁}V_{f}+P_{PFC}_{₂}(1−V_{f}), where P_{PFC}_{₁} and P_{PFC}_{₂} are the activation threshold for the two perfluorocarbons used (in this case perfluorohexane and perfluorobutane), and V_{f} represents the volume fraction of perfluorohexane. Using the linear fit an R-squared of 0.975 was obtained.

FIG. 7A is a photograph of an ultrasound image of a spinal cord of a rat model imaged using a 15 MHz linear array prior to injection of a nanoemulsion of the present disclosure. Harmonic imaging revealed no contrast, confirming the absence of bubbles.

FIG. 7B is a photograph of an ultrasound image of the spinal cord of a rat model, after a bolus injection of perfluorobutane nanodroplets active in the region of interest, highlighted using plane-wave harmonic imaging. Seconds after injection, individual activated droplets are seen passing through the spinal cord vasculature.

FIG. 7C is a photograph of an ultrasound image of the spinal cord of a rat model, after a bolus injection of perfluorobutane droplets active in the region of interest, highlighted using plane-wave harmonic imaging in conjunction with conventional ultrasound imaging. Seconds after injection, individual activated droplets are seen passing through the spinal cord vasculature.

FIG. 7D is a photograph of an ultrasound image of the spinal cord of a rat model after a bolus injection of perfluorobutane droplets active in the region of interest, after performing a maximum intensity projection of perfusing activated droplets (i.e., bubbles), microvessels were traced out over the image region.

FIG. 8 is a graph of zeta potential measurements of PFC droplets with and without a lipid stabilizer. Error bars represent one standard deviation.

FIG. 9 is a graph of short time scale changes in PFC droplet size with and without a lipid stabilizer.

FIG. 10A is graph showing dynamic light scattering measurements of perfluorocarbon nanodroplets produced with a lipid stabilizer.

FIG. 10B is a graph showing dynamic light scattering measurements of perfluorocarbon nanodroplets produced with an albumin stabilizer.

### DETAILED DESCRIPTION

The present disclosure features, inter alia, a kit, including a first compartment including a volatile fluorinated compound dissolved in a C₁₋₆ alcohol; and a second compartment including water. The volatile fluorinated compound is present in the first compartment at a concentration of 0.2% or more and 4% or less by volume, and the volume ratio of the volatile fluorinated compound and C₁₋₆ alcohol:water is from 24:1 to 500:1. As used herein, a recited range includes the end points, for example, a range of from 0.2% to 4% by volume includes both end points of 0.2% and 4% by volume. When the contents of the first and second compartments are mixed, spontaneous nucleation of nanodroplets of the volatile fluorinated compound can form in the aqueous phase to provide a dispersion of nanodroplets in the aqueous phase, also referred to herein as a nanoemulsion or a nanodroplet composition.

In some embodiments, the volatile fluorinated compound is present in the first compartment at a concentration of 0.2% or more (e.g., 0.5% or more, 1% or more, 2% or more, or 3% or more) and/or 4% or less (e.g., 3% or less, 2% or less, 1% or less, or 0.5% or less) by volume. For example, the volatile fluorinated compound can be present in the first compartment at a concentration of from 0.2% to 4% by volume (e.g., 0.5% to 4% by volume, 1% to 4% by volume, 1% to 3% by volume, or 2% to 4% by volume).

In some embodiments, the volume ratio of the volatile fluorinated compound and C₁₋₆ alcohol:water is 24:1 or more (e.g., 50:1 or more, 100:1 or more, 200:1 or more, 300:1 or more, or 400:1 or more) and/or 500:1 or less (e.g., 400:1 or less, 300:1 or less, 200:1 or less, 100:1 or less, or 50:1 or less). For example, the volume ratio of the volatile fluorinated compound and C₁₋₆ alcohol:water can be from 24:1 to 500:1 (e.g., from 50:1 to 500:1, from 100:1 to 500:1, from 100:1 to 400:1, from 100:1 to 300:1, or from 200:1 to 500:1).

The volatile fluorinated compound is present in the first compartment at a concentration of 0.01 M (molar) or more (e.g., 0.05M or more, 0.1M or more, 0.2M or more, 0.3M or more) and/or and 0.34 M or less (e.g., 0.3M or less, 0.2M or less, 0.1M or less, 0.05M or less).

The first compartment can include one or more volatile fluorinated compounds.

In some embodiments, the first and the second compartments are within a container, or together form a single container. The first and second compartments in the kit are not in fluid communication with one another. The two compartments can be separated by an impermeable barrier that can be broken (e.g., that is frangible) by a user to mix the contents of the two compartments. In some embodiments, a seal separates the two compartments. The seal can be broken, for example, by the user prior to administration to a subject, thereby generating a nanoemulsion including nanodroplets of volatile fluorinated compound dispersed in an aqueous phase. In some embodiments, the first and the second compartments are each a free-standing container, which the user can open and then mix the contents together to generate a nanoemulsion.

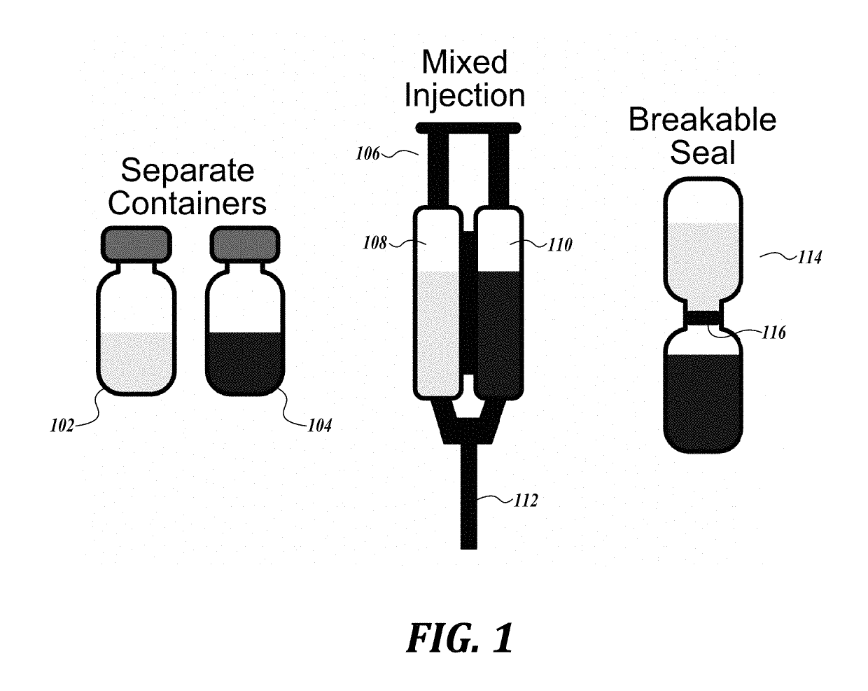

Examples of kits having first and second compartments are illustrated in FIG. 1. Referring to FIG. 1, the first and second compartments can be separate containers 102 and 104; or be present in a syringe 106 having two chambers 108 and 110, wherein one chamber includes a volatile fluorinated compound dissolved in a C₁₋₆ alcohol; and a second chamber includes water, and the contents of the two chambers can be mixed when the plunger pushes the contents into a single nozzle 112 (e.g., in preparation for injection); or the two compartments can form a single container 114 having a breakable seal 116 separating the contents 118 and 120 of the two compartments.

In some embodiments, either or both of the compartments further include a nanodroplet stabilizer. The stabilizer can coat the nanodroplets to form a shell around the nanodroplets. The stabilizer can be, for example, a lipid, a protein, a polymer, or any combination thereof. In some embodiments, the stabilizer does not include one or more of polyvinyl alcohol, polypyrrole, and pyrrole. Examples of a stabilizer include dipalmitoylphosphatidylcholine, 1,2-distearoyl-sn-glycero-3-phosphocholine, 1,2-distearoyl-sn-glycero-3-phosphoethanolamine, 1,2-dipalmitoyl-sn-glycero-3-phosphate, albumin, lysozyme, polyethylene glycol stearate, poly(D,L-lactide-co-glycolide), polylactic acid, and/or polyvinyl alcohol. In some embodiments, the stabilizer is dipalmitoylphosphatidylcholine, 1,2-distearoyl-sn-glycero-3-phosphocholine, 1,2-distearoyl-sn-glycero-3-phosphoethanolamine, 1,2-dipalmitoyl-sn-glycero-3-phosphate, albumin, lysozyme, polyethylene glycol stearate, poly(D,L-lactide-co-glycolide), and/or polylactic acid. The stabilizer can be present in a concentration of 0.4 pM or more (e.g., 1 pM or more, 10 pM or more, 100 pM or more, 1 nM or more, 10 nM or more, 100 nM or more, 1 μM or more, 10 μM or more, 100 μM or more, 1 mM or more, 10 mM or more, or 100 mM or more) and/or 200 mM or less (e.g., 100 mM or less, 10 mM or less, 1 mM or less, 100 μM or less, 10 μM or less, 1 μM or less, 100 nM or less, 10 nM or less, 1 nM or less, 100 pM or less, 10 pM or less, or 1 pM or less), relative to the total contents of the first and second compartments, or relative to the resulting nanoemulsion.

In some embodiments, either or both of the compartments further include a therapeutic agent. Examples of therapeutic agents include doxorubicin, chlorambucil, t-PA (tissue plasminogen activator), serine proteases, and/or plasmids. In some embodiments, the therapeutic agents are used for cancer therapy, thrombolysis, and/or gene therapy. In some embodiments, either or both of the compartments further include a cell-targeting agent. For example, the cell-targeting agent can include peptides, ligands, integrins, synthetic polymers, and porphyrins such as cyclic RGD, αVβ3 integrin, CREKA peptide, fibrin binding peptide, verteporfin, P-selectin, intercellular adhesion molecule 1, and/or vascular cell adhesion molecule 1. The therapeutic agent can be present in a concentration of 0.01 pM or more (e.g., 0.1 pM or more, 1 pM or more, 10 pM or more, 100 pM or more, 1 nM or more, 10 nM or more, 100 nM or more, 1 μM or more, 10 μM or more, 100 μM or more, 1 mM or more, 10 mM or more, 100 mM or more, or 250 mM or more) and/or 500 mM or less (e.g., 250 mM or less, 100 mM or less, 10 mM or less, 1 mM or less, 100 μM or less, 10 μM or less, 1 μM or less, 100 nM or less, 10 nM or less, 1 nM or less, 100 pM or less, 10 pM or less, 1 pM or less, or 0.1 pM or less), relative to the total contents of the first and second compartments, or relative to the resulting nanoemulsion. The cell-targeting agent can be present in a concentration of 0.01 pM or more (e.g., 0.1 pM or more, 1 pM or more, 10 pM or more, 100 pM or more, 1 nM or more, 10 nM or more, 100 nM or more, or 1 μM or more) and/or 10 μM or less (1 μM or less, 100 nM or less, 10 nM or less, 1 nM or less, 100 pM or less, 10 pM or less, 1 pM or less, or 0.1 pM or less), relative to the total contents of the first and second compartments, or relative to the resulting nanoemulsion.

In some embodiments, the C₁₋₆ alcohol is methanol or ethanol. In certain embodiments, the C₁₋₆ alcohol is ethanol.

In some embodiments, the volatile fluorinated compound is sulfur hexafluoride and/or a perfluorocarbon. In some embodiments, the perfluorocarbon is perfluorohexane, perfluoropentane, perfluorobutane, and/or perfluoropropane. As used herein, a volatile fluorinated compound refers to a compound that can evaporate at a room temperature of around 20° C. at 1 atm pressure, or that is gaseous at a room temperature of around 20° C. at 1 atm pressure. Without wishing to be bound by theory, it is believed that the volatile fluorinated compound is in a liquid state when in the form of a nanodroplet in a nanoemulsion at room temperature and 1 atm pressure due to capillary forces, even when the volatile fluorinated compound would otherwise be in a gaseous form at a room temperature of around 20° C. at 1 atm pressure.

In some embodiments, either or both of the compartments can further include a buffering agent (e.g., a phosphate buffer), a co-solvent with water (e.g., a polar water-miscible solvent, such as glycerol and/or propylene glycol), a salt (e.g., sodium chloride and/or potassium chloride), and/or a sugar (e.g., dextrose). In certain embodiments, the second compartment further includes a buffering agent. The buffering agent can be present in a concentration of 0.01 M or more (e.g., 0.1 M or more, 0.25 M or more, 0.5 M or more, or 0.75 M or more) and/or 1 M or less (e.g., 0.75 M or less, 0.5 M or less, 0.25 M or less, or 0.1 M or less), relative to the total contents of the first and second compartments, or relative to the resulting nanoemulsion. The co-solvent can be present in a concentration of 25% or more (e.g., 40% or more, 50% or more, 60% or more, 70% or more, 80% or more, or 90% or more) and/or 99% or less (e.g., 90% or less, 80% or less, 70% or less, 60% or less, 50% or less, or 40% or less) by volume, relative to the total contents of the first and second compartments, or relative to the resulting nanoemulsion. The salt can be present in a concentration of 30 mM or more (e.g., 100 mM or more, 250 mM or more, 500 mM or more, 750 mM or more, or 1M or more) and/or 1.2 M or less (e.g., 1M or less, 750 mM or less, 500 mM or less, 250 mM or less, or 100 mM or less), relative to the total contents of the first and second compartments, or relative to the resulting nanoemulsion. In some embodiments, the salt can be present in a concentration of from 30 mM to 300 mM (e.g., about 155 mM) relative to the total contents of the first and second compartments, or relative to the resulting nanoemulsion.

In some embodiments, the volatile fluorinated compound is present in a concentration of from 0.01% to 3.5% by volume; the stabilizer (e.g., a surfactant) is present in a concentration of from 1% to 7.5% by volume; and water is preset in a concentration of from 21.5% to 98.99%, relative to the total contents of the first and second compartments, or relative to the resulting nanoemulsion.

### Nanoemulsions and Methods of Making Nanoemulsions

The present disclosure also features, inter alia, a method of making nanoemulsions of nanodroplets of volatile fluorinated compounds that are uniformly dispersed in an aqueous phase. The method includes dissolving a volatile fluorinated compound in a good solvent for the fluorinated compound (e.g. a C₁-C₆ alcohol, such as ethanol), a second poor solvent (e.g. water) is then added to the solution that includes the dissolved volatile fluorinated compound, which drives the dissolved volatile fluorinated compound out of solution, resulting in spontaneously nucleated nanodroplets. The volatile fluorinated compound in a good solvent can be contained, for example, in a first compartment of the kit described above. The poor solvent can be contained, for example, in a second compartment of the kit described above. In some embodiment, a stabilizer is included in the good and/or poor solvent to help stabilize the nanodroplets of volatile fluorinated compound after droplet nucleation. In some embodiments, the nanoemulsion does not include one or more of polyvinyl alcohol, polypyrrole, and pyrrole.

The nanoemulsions of the present disclosure can have nanodroplets that are stable for an extended duration, such that the nanodroplets maintain their morphology and size over an extended time period. For example, the nanodroplets in the nanoemulsion can be stable for 2 days or more (e.g., 5 days or more, 1 week or more, 2 weeks or more, 3 weeks or more, 4 weeks or more, or 5 weeks or more) and/or 6 weeks or less (e.g., 5 weeks or less, 4 weeks or less, 3 weeks or less, 2 weeks or less, 1 week or less, or 5 days or less), when the nanoemulsion is stored at room temperature. When the nanoemulsion is stored at 4° C., the stability of the nanodroplets can be additionally enhanced compared to room temperature storage. For example, at 4° C., the nanodroplets in the nanoemulsion can be stable for one week or more (e.g., 2 weeks or more, 4 weeks or more, or 6 weeks or more) and/or 8 weeks or less (e.g., 6 weeks or less, 4 weeks or less, or 2 weeks or less).

The nanodroplets can be isolated. For example, when the nanodroplets are stabilized with a stabilizer, the nanoemulsions can be centrifuged and/or dialyzed, and the stabilized nanodroplets of the volatile fluorinated compound can be separated and isolated from the aqueous liquid in which they are suspended. The isolated nanodroplets can be reconstituted for later use by resuspending in an aqueous solution, such as saline, to provide a nanoemulsion.

The nanodroplets can have an average diameter of 60 nm or more (e.g., 100 nm or more, 250 nm or more, 500 nm or more, 750 nm or more, 1 μm or more, 1.25 μm or more, 1.5 μm or more, or 1.75 μm or more) and/or 2 μm or less (e.g., 1.75 μm or less, 1.5 μm or less, 1.25 μm or less, 1 μm or less, 750 nm or less, 500 nm or less, 250 nm or less, or 100 nm or less). In some embodiments, the nanodroplets have an average diameter of from 60 nm to 700 nm. The dispersion of nanodroplets in the nanoemulsion can have an average droplet volume of 1.1×10⁵ nm³ or more (e.g., 1×10⁶ nm³ or more, 1×10⁷ nm³ or more, or 1×10⁸ nm³ or more) and/or 4.2×10⁹ nm³ or less (e.g., 1×10⁸ nm³ or less, 1×10⁷ nm³ or less, or 1×10⁶ nm³ or less). For example, the nanodroplets can have an individual average droplet volume of from 1.1×10⁵ nm³ to 4.2×10⁹ nm³ (e.g., from 1.1×10⁵ nm³ to 1×10⁸ nm³, from 1.1×10⁵ nm³ to 1×10⁷ nm³, from 1.1×10⁵ nm³ to 1×10⁶ nm³, from 1×10⁶ nm³ to 4.2×10⁹ nm³, from 1×10⁷ nm³ to 4.2×10⁹ nm³, or from 1×10⁸ nm³ to 4.2×10⁹ nm³). The nanodroplet size distribution can be determined using dynamic light scattering (DLS). Without wishing to be bound by theory, DLS measures droplets sizes by monitoring the time scale of fluctuations in light transmitted or reflected from the droplets. The high frequency fluctuations in the light are correlated with the Brownian motion of smaller droplets. Accordingly, the average diameter and/or volume of nanodroplets, nanobubbles, or microbubbles of the present disclosure can be assessed by measuring the diameters and/or volume of the nanodroplets, nanobubbles, or microbubbles using dynamic light scattering, and the averages can be based on 5 samples per condition with 3 size measurements per sample. In some embodiments, Coulter counters and flow cytometers can be used for droplets having a diameter of greater than or equal to 1 μm.

In some embodiments, the volume of the nanodroplets in a nanoemulsion can be relatively uniform. For example, the nanodroplets in a nanoemulsion can have a polydispersity index (PDI=standard deviation/mean “σ/μ”) of 0.2 or less. In some embodiments, the nanodroplets in a nanoemulsion have a PDI of from 0.02 to 0.2 (e.g., from 0.04 to 0.2, from 0.08 to 0.2, from 0.1 to 0.2, from 0.1 to 0.15, or about 0.1). As an example, in DLS measurements the droplet distribution can be a single peak with a PDI (standard deviation/mean) of about 0.1. Varying the relative concentration of the volatile fluorinated compound, the good solvent (e.g., ethanol), and poor solvent (e.g., water) can determine the resulting droplet size. For a PDI of about 0.1, a mean droplet diameter of about 200 nm can have a standard deviation of about 20 nm, which corresponds to a volumetric range of about 2.4×10⁷ nm³ to 4.5×10⁷ nm³, where the mean is about 3.4×10⁷ nm³.

The nanodroplets can be present in the aqueous solution or nanoemulsion at a concentration of 1×10⁷ or more (e.g., 1×10⁸ or more, 1×10⁹ or more, 1×10¹⁰ or more, 1×10¹¹ or more, 1×10¹² or more, or 1×10¹³ or more) and/or 1×10¹⁴ or less (e.g., 1×10¹³ or less, 1×10¹² or less, 1×10¹¹ or less, 1×10¹⁰ or less, 1×10⁹ or less, or 1×10⁸ or less) nanodroplets per ml of aqueous solution or nanoemulsion. In some embodiments, the nanodroplets are present in the aqueous solution or nanoemulsion at a concentration of 1×10⁷ to 1×10¹⁴ (e.g., 1×10⁷ to 1×10¹³, 1×10⁷ to 1×10¹², 1×10⁷ to 1×10¹¹, 1×10⁷ to 1×10¹⁰, 1×10⁷ to 1×10⁹, 1×10⁷ to 1×10⁸, 1×10⁸ to 1×10¹⁴, 1×10⁹ to 1×10¹⁴, 1×10¹⁰ to 1×10¹⁴, 1×10¹¹ to 1×10¹⁴, 1×10¹² to 1×10¹⁴, or 1×10¹³ to 1×10¹⁴ nanodroplets per ml of aqueous solution or nanoemulsion.

In some embodiments, the nanoemulsion further includes a nanodroplet stabilizer. The stabilizer can coat the nanodroplets to form a shell around the nanodroplets. The stabilizer can be, for example, a lipid, a protein, a polymer, or any combination thereof. In some embodiments, the stabilizer does not include one or more of polyvinyl alcohol, polypyrrole, and pyrrole. Examples of a stabilizer include dipalmitoylphosphatidylcholine, 1,2-distearoyl-sn-glycero-3-phosphocholine, 1,2-distearoyl-sn-glycero-3-phosphoethanolamine, 1,2-dipalmitoyl-sn-glycero-3-phosphate, albumin, lysozyme, polyethylene glycol stearate, poly(D,L-lactide-co-glycolide), polylactic acid, and/or polyvinyl alcohol. In some embodiments, the stabilizer is dipalmitoylphosphatidylcholine, 1,2-distearoyl-sn-glycero-3-phosphocholine, 1,2-distearoyl-sn-glycero-3-phosphoethanolamine, 1,2-dipalmitoyl-sn-glycero-3-phosphate, albumin, lysozyme, polyethylene glycol stearate, poly(D,L-lactide-co-glycolide), and/or polylactic acid. The stabilizer can be present in a concentration of 0.4 pM or more (e.g., 1 pM or more, 10 pM or more, 100 pM or more, 1 nM or more, 10 nM or more, 100 nM or more, 1 μM or more, 10 μM or more, 100 μM or more, 1 mM or more, 10 mM or more, or 100 mM or more) and/or 200 mM or less (e.g., 100 mM or less, 10 mM or less, 1 mM or less, 100 μM or less, 10 μM or less, 1 μM or less, 100 nM or less, 10 nM or less, 1 nM or less, 100 pM or less, 10 pM or less, or 1 pM or less), relative to the total contents of the nanoemulsion.

In some embodiments, the nanoemulsion further includes a therapeutic agent and/or a cell-targeting agent, as discussed above. The therapeutic agent can be present in the nanoemulsion at a concentration of 0.01 pM or more (e.g., 0.1 pM or more, 1 pM or more, 10 pM or more, 100 pM or more, 1 nM or more, 10 nM or more, 100 nM or more, 1 μM or more, 10 μM or more, 100 μM or more, 1 mM or more, 10 mM or more, 100 mM or more, or 250 mM or more) and/or 500 mM or less (e.g., 250 mM or less, 100 mM or less, 10 mM or less, 1 mM or less, 100 μM or less, 10 μM or less, 1 μM or less, 100 nM or less, 10 nM or less, 1 nM or less, 100 pM or less, 10 pM or less, 1 pM or less, or 0.1 pM or less). The cell-targeting agent can be present in the nanoemulsion at a concentration of 0.01 pM or more (e.g., 0.1 pM or more, 1 pM or more, 10 pM or more, 100 pM or more, 1 nM or more, 10 nM or more, 100 nM or more, or 1 μM or more) and/or 10 μM or less (1 μM or less, 100 nM or less, 10 nM or less, 1 nM or less, 100 pM or less, 10 pM or less, 1 pM or less, or 0.1 pM or less). In some embodiments, the targeting agent is located on the surface of a nanodroplet. In some embodiments, the therapeutic agent is located on the surface of the nanodroplet, within the nanodroplet, or among the stabilizer covering the surface of the nanodroplet. In some embodiments, the nanodroplets can include secondary diagnostic agents (e.g., gadolinium, barium, iodine, and compounds including gadolinium, barium, or iodine) for combined ultrasound and MRI, X-ray or CT imaging.

In some embodiments, the C₁₋₆ alcohol in the nanoemulsion is methanol or ethanol. In certain embodiments, the C₁₋₆ alcohol is ethanol.

In some embodiments, the volatile fluorinated compound is sulfur hexafluoride and/or a perfluorocarbon. In some embodiments, the perfluorocarbon is perfluorohexane, perfluoropentane, perfluorobutane, and/or perfluoropropane. As used herein, a volatile fluorinated compound refers to a compound that evaporates at a room temperature of around 20° C. at 1 atm pressure, or that is gaseous at a room temperature of around 20° C. at 1 atm pressure. Without wishing to be bound by theory, it is believed that the volatile fluorinated compound is in a liquid state when in the form of a nanodroplet in a nanoemulsion at room temperature and 1 atm pressure, even when the volatile fluorinated compound would otherwise be in a gaseous form at a room temperature of around 20° C. at 1 atm pressure, due to capillary forces.

The nanoemulsion can include one or more volatile fluorinated compounds.

In some embodiments, the nanoemulsion can further include a buffering agent (e.g., a phosphate buffer), a co-solvent with water (e.g., a polar water-miscible solvent, such as glycerol and/or propylene glycol), a salt (e.g., sodium chloride or potassium chloride), and/or a sugar (e.g., dextrose). The buffering agent can be present in a concentration of 0.01 M or more (e.g., 0.1 M or more, 0.25 M or more, 0.5 M or more, or 0.75 M or more) and/or 1 M or less (e.g., 0.75 M or less, 0.5 M or less, 0.25 M or less, or 0.1 M or less) in the nanoemulsion. The co-solvent can be present in a concentration of 25% or more (e.g., 40% or more, 50% or more, 60% or more, 70% or more, 80% or more, or 90% or more) and/or 99% or less (e.g., 90% or less, 80% or less, 70% or less, 60% or less, 50% or less, or 40% or less) by volume in the nanoemulsion. The salt can be present in a concentration of 30 mM or more (e.g., 100 mM or more, 250 mM or more, 500 mM or more, 750 mM or more, or 1 M or more) and/or 1.2 M or less (e.g., 1 M or less, 750 mM or less, 500 mM or less, 250 mM or less, or 100 mM or less) in the nanoemulsion. In some embodiments, the salt can be present in a concentration of from 30 mM to 300 mM (e.g., about 155 mM) in the nanoemulsion.

Using a sufficiently high amplitude acoustic pulse (e.g., a peak negative pressure of from 0.2 MPa to 7 MPa), the droplets of the volatile fluorinated compound can vaporize to form gas bubbles roughly 5 times larger than the initial droplet, which can be used for ultrasound imaging contrast or therapy. The nanobubbles or microbubbles can have an average diameter of 120 nm or more (e.g., 200 nm or more, 300 nm or more, 500 nm or more, 1 μm or more, 3 μm or more, 5 μm or more, 7 μm or more, or 9 μm or more) and/or 10 μm or less (e.g., 9 μm or less, 7 μm or less, 5 μm or less, 3 μm or less, 1 μm or less, 500 nm or less, 300 nm or less, or 200 nm or less). For example, the nanobubbles or microbubbles can have an average diameter of from 120 nm to 10 μm (e.g., from 200 nm to 10 μm, from 500 nm to 10 μm, from 1 μm to 10 μm, from 3 μm to 10 μm, from 5 μm to 10 μm, from 1 μm to 5 μm, or from 1 μm to 3 μm). In some embodiments, the nanobubble has an average diameter of 300 nm or less (e.g., 200 nm or less, or 100 nm or less). In some embodiments, the nanobubbles or microbubbles have an average diameter of from 120 nm to 3.5 μm. The size of the bubble distribution can be measured using techniques such as dynamic light scattering, Coulter counter, or flow cytometry. Alternatively or in addition, the bubble size distribution can be calculated based off of the synthesized droplet diameter and molecular weight using the ideal gas law and Laplace's law.

The activation of the nanodroplets by ultrasound can occur in vivo and/or in vitro. Because of the expansion ratio of the nanodroplets of volatile fluorinated compound, nanodroplets having a diameter of 200 nm or less (small enough to diffuse past the vessel wall for extravascular applications) can be synthesized with little to no loss in echogenic properties.

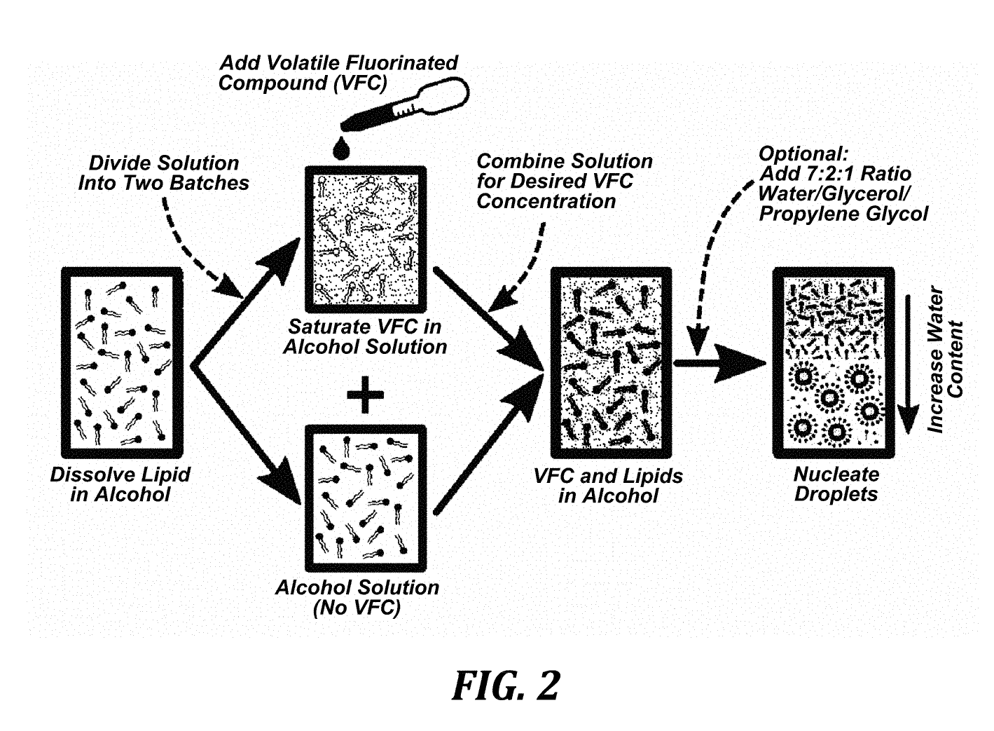

As an example, FIG. 2 shows a method of the present disclosure for nanodroplet production by spontaneous nucleation of liquid nanodroplets in solution. A lipid surfactant, or any other stabilizer, is first dissolved in ethanol. This solution is then divided such that only one solution is fully saturated with a volatile fluorinated compound (VFC). The fully saturated VFC solution and the pure lipid in ethanol solution are re-combined to achieve a desired VFC concentration in the solution. A water-based solution is then pipetted into the container with VFC and lipid dissolved in ethanol. Introducing water causes the VFC to lose solubility, leading to VFC nanodroplet nucleation.

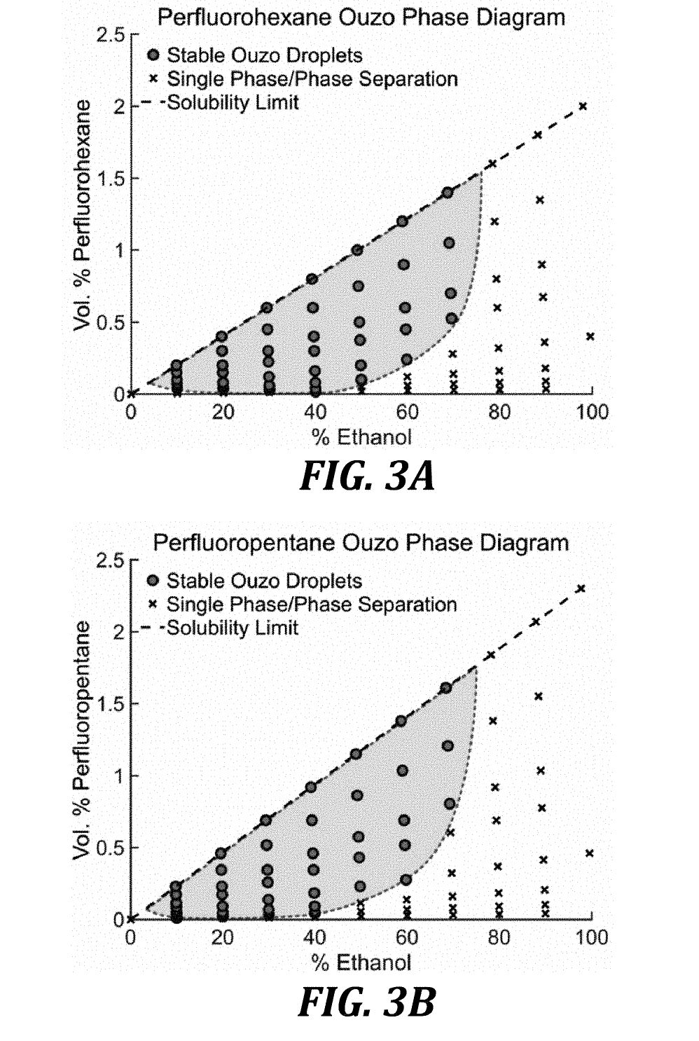

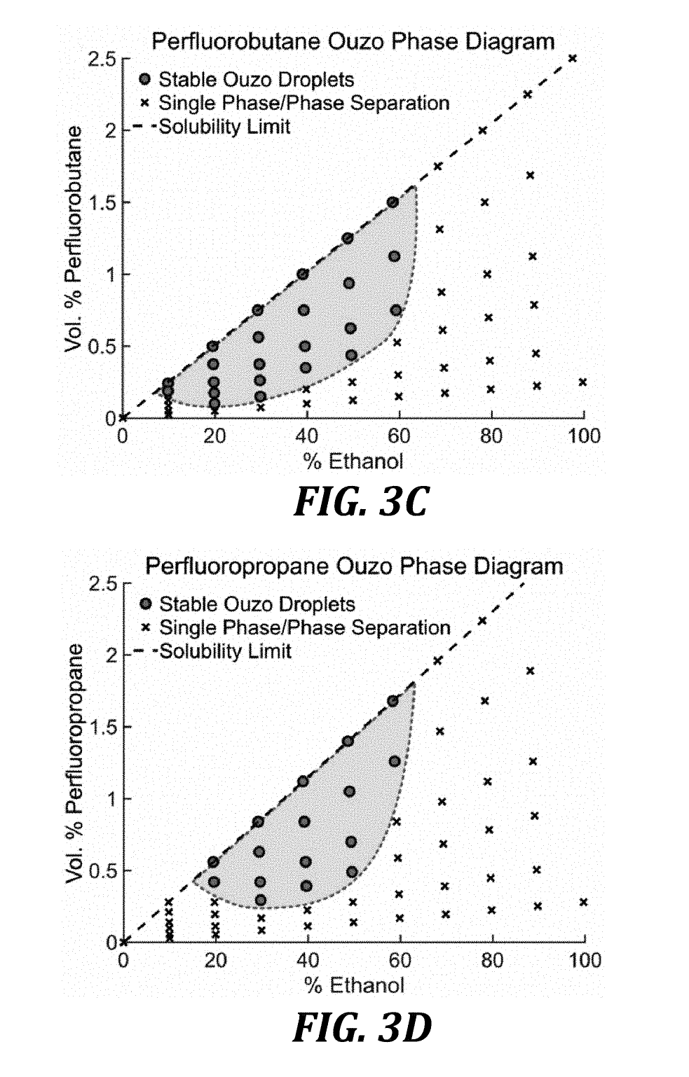

Referring to FIGS. 3A and 3D, the ouzo phase diagrams of various perfluorocarbons can indicate the conditions leading to droplet formation (e.g., ouzo regions showing in the shaded area with a dotted line border). Droplets can nucleate in a narrow region relative to the entire ternary diagram. Without wishing to be bound by theory, it is believed that while the ratio of concentrations in order to maintain the ouzo effect in ouzo is narrow, when using volatile fluorinated compounds, the range of concentrations allowing droplet nucleation is more forgiving. For example, sulfur hexafluoride, perfluorohexane, perfluoropentane, perfluorobutane, and perfluoropropane provide good droplet formation ranges, and are desirable to use as bubbles in a mammalian body due to their boiling points at atmospheric pressure and at pressures experienced within mammalian bodies, making the bubbles less likely to revert to a liquid phase once activated by ultrasound.

### Uses

When used in therapeutic, diagnostic, or theranostic settings, the nanodroplet composition (e.g., the nanoemulsions) of the present disclosure can be administered (e.g., by injection) to a subject (e.g., a mammal, a human) in need thereof. The administered nanodroplet composition can be exposed to ultrasound to vaporize the nanodroplets, thereby providing nanobubbles or microbubbles. In some embodiments, the nanodroplet composition does not comprise polyvinyl alcohol, polypyrrole, or pyrrole. As used herein, “theranostic” refers to describe procedures that integrate diagnostic and therapeutic components. For example, nanobubbles or microbubbles provided by the nanodroplet composition can be used to assist in the diagnosis of a condition by serving as an ultrasound contrast agent, and can provide localized therapy by delivering a therapeutic agent contained in the nanobubbles or microbubbles. The effectiveness of the therapy can then be monitored by ultrasound imaging of the nanobubbles or microbubbles.

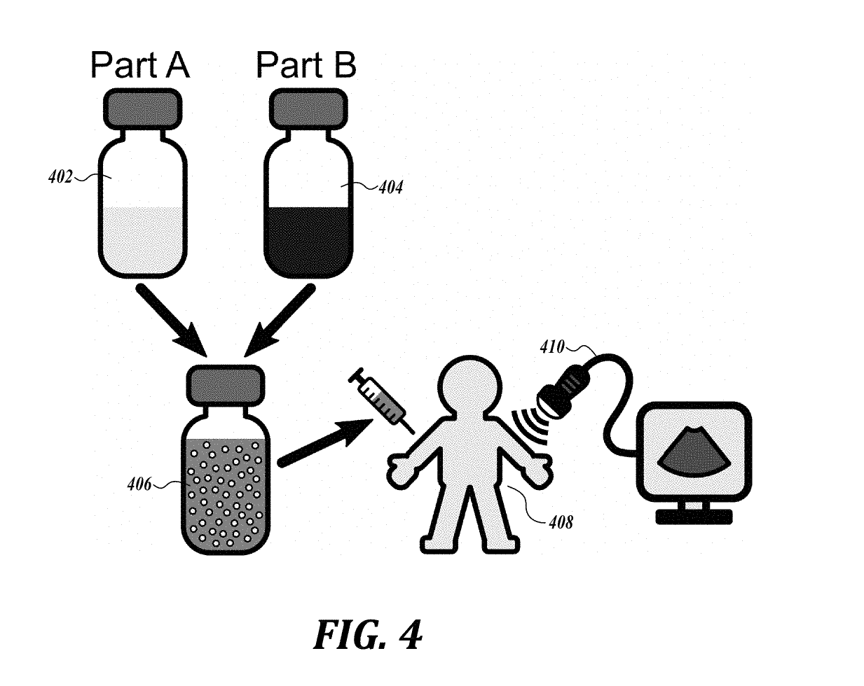

Referring to FIG. 4, in some embodiments, when kits of the present disclosure are provided, the user (such as a doctor, a nurse, or an ultrasound technician) can combine the contents of the first and second compartments 402 and 404 of the kit to provide the nanodroplet composition 406, prior to injecting the nanodroplet composition into a subject 408. Ultrasound 410 can then be applied to the subject for therapy and/or diagnosis.

In some embodiments, the nanoemulsion including the nanodroplets is used to deliver a therapeutic agent to a target location, such as a tumor, and can release the therapeutic agent at that location upon activation. The liquid nanodroplets can provide nanobubbles or microbubbles upon ultrasound exposure, which can then be further activated (e.g., via additional ultrasound exposure) to produce a cavitation effect, and ablate target tissues in a histotripsy application.

In some embodiments, when the nanodroplets are introduced to the vascular system, the nanodroplets can target a specific location, such as a tumor or other tissue location, and can pass through vascular walls and into tissue. In some embodiments, the nanodroplets can have a diameter of less than about 100 nm. In some embodiments, the liquid droplets, when exposed to ultrasound, provide contrast for ultrasound imaging in medical applications. In some embodiments, the liquid nanodroplets, when activated, can also or alternatively serve as a therapeutic agent.

Example devices, methods, and systems are described herein. In should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the FIGURES should not be viewed as limiting. It should be understood that other embodiments may include more or less of each element shown in a given FIGURE. Further, some of the illustrated elements may be combined or omitted. Yet further, an example embodiment may include elements that are not illustrated in the FIGURES. As used herein, with respect to measurements, “about” means +/−5%.

### EXAMPLES

### Example 1. Spontaneous Nucleation of Stable Perfluorocarbon Emulsions for Ultrasound Contrast Agents

The present example describes a method of producing stable nanodroplets of liquid PFCs in solution, that in turn, when activated in vivo by ultrasound, produce a more uniform and stable bubble for imaging and therapy than current FDA approved methods.

Phase-change contrast agents are rapidly developing as an alternative to microbubbles for ultrasound imaging and therapy. These agents are synthesized and delivered as liquid droplets and vaporized locally to produce image contrast. They can be used like conventional microbubbles but with the added benefit of reduced size and improved stability. Droplet-based agents can be synthesized with diameters on the order of 100 nm, making them an ideal candidate for extravascular imaging or therapy. However, their synthesis requires low boiling point perfluorocarbons (PFCs) to achieve activation (i.e., vaporization) thresholds within FDA approved limits. Minimizing spontaneous vaporization while producing liquid droplets using conventional methods with low boiling point PFCs can be challenging. In this study, a new method to produce PFC nanodroplets using spontaneous nucleation is demonstrated using PFCs with boiling points ranging from −37 to 56° C. Sometimes referred to as the ouzo method, the process relies on saturating a cosolvent with the PFC before adding a poor solvent to reduce solvent quality, forcing droplets to spontaneously nucleate. This approach can produce droplets ranging from under 100 nm to over 1 μm in diameter. Ternary plots showing solvent and PFC concentrations leading to droplet nucleation are presented. Additionally, acoustic activation thresholds and size distributions with varying PFC and solvent conditions are measured and discussed. Finally, ultrasound contrast imaging is demonstrated using ouzo droplets in an animal model.

In this Example, an alternative method of synthesizing PFC nanodroplets through spontaneous droplet nucleation is demonstrated. In general, the method (referred to herein as the ouzo method) has two steps beginning with dissolving the oil (PFC) into a “good” cosolvent (alcohol), which is also completely miscible with a “poor” solvent (water). To nucleate the droplets, the “poor” solvent is added to the dissolved oil in the “good” cosolvent. By adding water to the PFC/alcohol solution, the oil solubility is rapidly reduced, forcing the oil phase out of solution and spontaneously nucleating droplets with surprising monodispersity and stability. In this study, ternary phase diagrams showing the solvent conditions necessary to nucleate PFC droplets are presented. In addition, droplet size distribution, stability, and activation thresholds for contrast agents are discussed. Finally, ultrasound imaging using ouzo PFC droplets is demonstrated in an animal model.

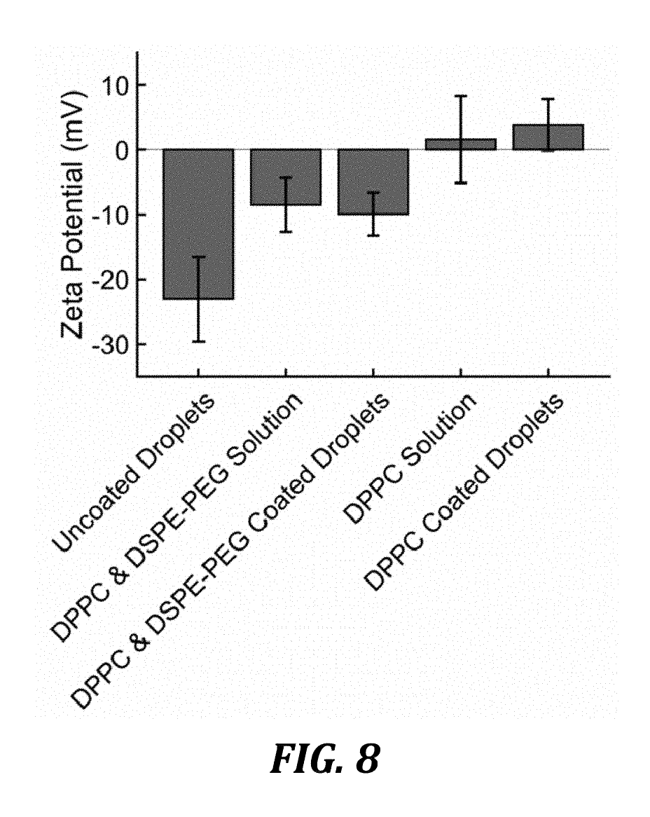

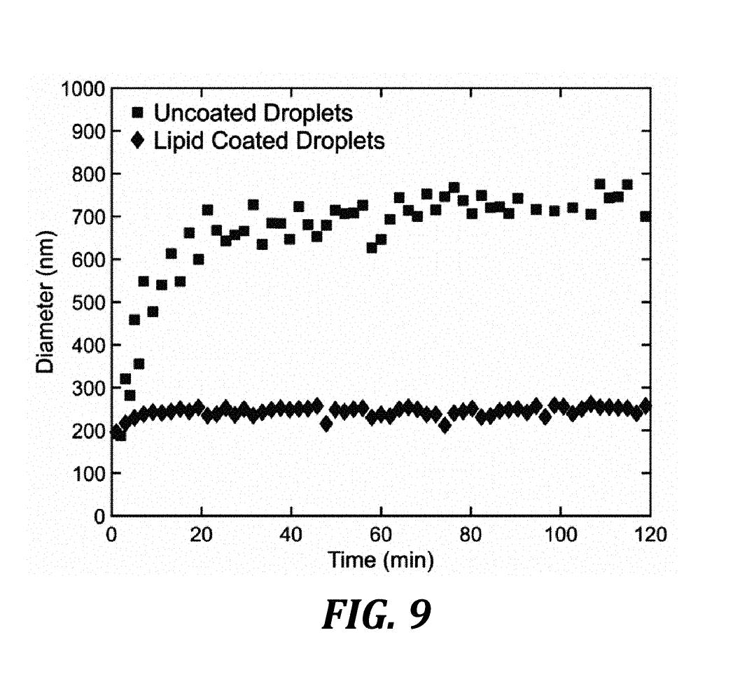

PFC ouzo droplets were synthesized by first dissolving a PFC into ethanol (co-solvent). A maximum of approximately 2.0% perfluorohexane (C₆F₁₄, T_{Boiling}=56° C.), 2.3% perfluoropentane (C₅F₁₂, T_{Boiling}=29° C.), 2.5% perfluorobutane (C₄F₁₀, T_{Boiling}=2° C.), and 2.7% perfluoropropane (C₈F₈, T_{Boiling}=−37° C.) by volume could be dissolved in ethanol. In the ethanol phase, a 20:1 mole ratio of dipalmitoylphosphatidylcholine (DPPC) and N-(Methylpolyoxyethylene oxycarbonyl)-1,2-distearoyl-sn-glycero-3-phosphoethanolamine sodium salt (DSPE-PEG 2K) lipids were also dissolved. The total lipid concentration was varied from 0.10 mg/ml to 1.82 mg/ml depending on the volume of perfluorocarbon that was used. Lipid concentration much less 0.10 mg/ml still resulted in stable emulsions. However, lower concentration lipids solutions were not used to ensure ample lipids were in solution to stabilize the droplet interface. Droplet formulations using greater lipid concentration in solution resulted in the formation of lipid aggregates and destabilizing droplets through flocculation. When an aqueous solution (solvent) prepared at a ratio of 7:2:1 water/propylene glycol/glycerol is added to the PFC/ethanol solution, the PFC oil phase quickly loses solubility resulting in the spontaneous nucleation of stable droplets (FIG. 2). Since PFCs are known to have very poor solubility in water, the greater the water content relative to ethanol, the lower the PFC solubility is in the final mixed solvent. The inclusion of glycerol and propylene glycol is optional for droplet nucleation. For example, PFC emulsions can also be generated in the absence of any stabilizer (FIGS. 8 and 9). However, glycerol and propylene glycol are commonly used along with lipid mixtures to improve microbubble stability. Here, glycerol and propylene glycol were also included in the ouzo droplet formulation to make the agent composition analogous to microbubbles and to increase stability.

Referring to FIG. 2, the ouzo method for volatile fluorinated compound (here, a perfluorocarbon, PFC) nanodroplet production is a process for the spontaneous nucleation of liquid nanodroplets in solution. A lipid surfactant, or any other stabilizer, is first dissolved in an alcohol, such as ethanol. This solution is then divided such that only one solution is fully saturated with perfluorocarbon. The fully saturated PFC solution and the pure lipid in alcohol (e.g., ethanol) solution are re-combined to achieve a desired PFC concentration in the solution. Finally, a water-based solution is pipetted into the container with PFC and lipid dissolved in alcohol (e.g., ethanol). Introducing water causes the PFC to lose solubility, leading to PFC nanodroplet nucleation. This process can be performed with any perfluorocarbon gas or liquid in combination with any stabilizer, such as a surfactant or shell coating.

A ternary phase diagram indicating the volume percentages of PFC oil, ethanol, and water solution (%_{Water}=100%−%_{Ethanol}−%_{PFC}) that is required to nucleate droplets was created for each of the PFCs tested (FIGS. 3A-3D). For all PFCs, a minimum water concentration was required to nucleate droplets. As the PFC concentration was reduced, a proportionally greater concentration of water was required to nucleate PFC droplets. Comparing the conditions using different PFCs, the size of the ouzo region for increasingly volatile PFCs (i.e. PFH to OFB) decreased. The reduced ouzo region in the ternary plot is likely due to an increase in PFC oil solubility in both ethanol and water with decreasing PFC molecular weight (i.e. increased volatility). This is also supported by the thermodynamics of cavity formation. Because larger molecular weight PFCs displace a larger number of water molecules than an equal number of a lower molecular weight PFC, the solvent conditions are less favorable for large molecular weight PFCs to stay in solution than low molecular weight ones. This results in the larger molecular weight PFCs having a larger ouzo region than the lower molecular weight PFCs.

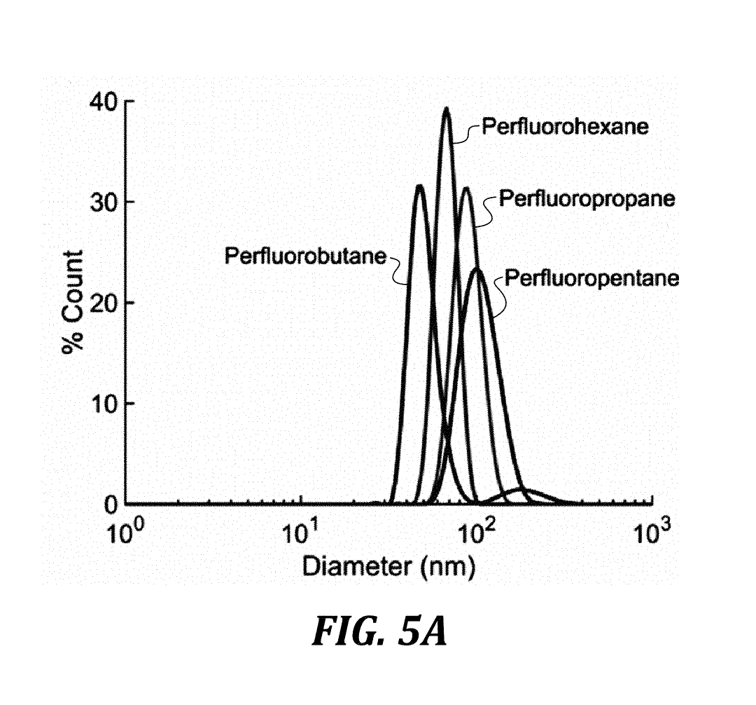

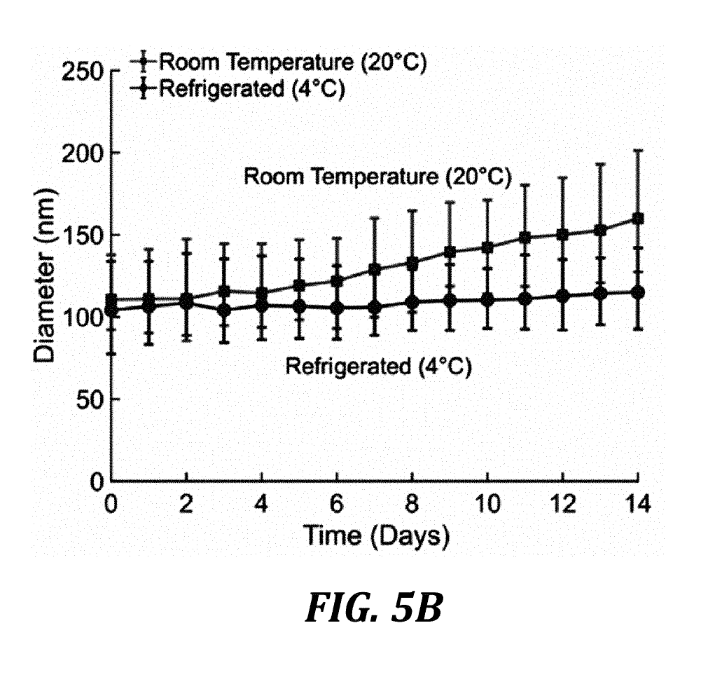

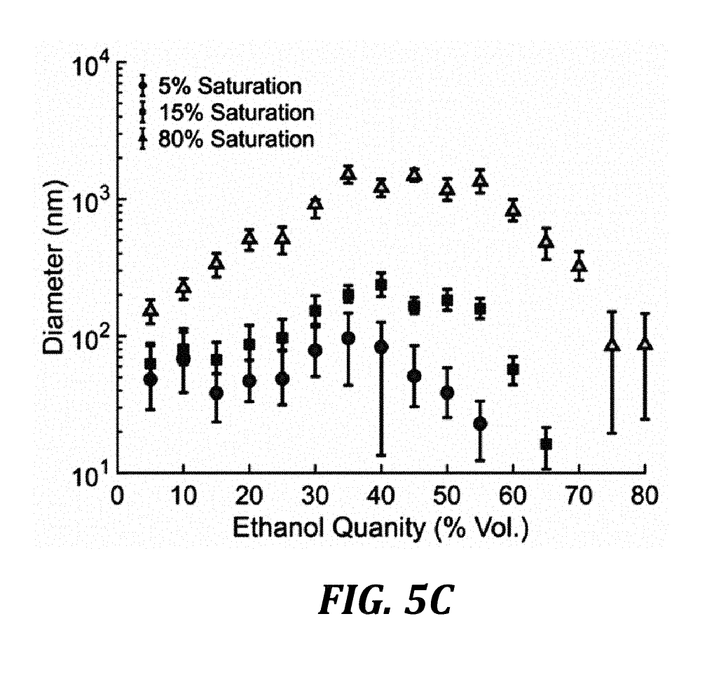

Although the PFC type also plays a small role in determining droplet size, the size distributions were primarily correlated to the PFC concentration and the ethanol/water ratio used to induce spontaneous emulsification (FIG. 5C). Nanodroplets with diameters on the order of 100 nm could be easily synthesized using all PFCs tested (FIG. 5A). Moreover, ouzo-synthesized droplets of about 100 nm in diameter were found to be stable for days or weeks depending on the storage conditions (FIG. 5B).

Zeta potential measurements also support that lipids successfully coated the droplet interface as they do with microbubbles (FIG. 8). The zeta potential increased from −23 mV for uncoated droplets to ˜9.93 mV for DPPC and DPSE-PEG-coated droplets and 3.9 mV for DPPC-coated droplets. Prior to measuring the zeta potential of the lipid-coated droplets, the samples were centrifuged to remove excess lipids in solution and redispersed. This process was repeated three times to ensure all excess lipids were removed and the zeta potential measurements were of the droplets. The zeta potentials of both lipid-coated droplets were statistically different from that of uncoated ones. Moreover, lipid-coated droplets matched the zeta potentials of lipid micelle samples, suggesting that the droplets were coated with lipids. In the absence of the lipid stabilizer, DLS measurements revealed a rapid increase in size in the droplet distribution, which was not seen with lipid-coated droplets (FIG. 9). The increase in uncoated droplet size was likely due to a combination of coalescence in the short time scale and Ostwald ripening over the longer time scales. In contrast, lipid-coated droplets had nearly no change in size throughout the same time scale, suggesting that lipids can decrease unwanted coalescence.

Perfluorohexane was also used as a representative PFC to further investigate changes in droplet size distribution as a function of oil concentration and relative ethanol-to-water ratios (FIG. 5C). In general, as PFC saturation in ethanol increased, the average droplet size also increased. This result was expected because a solution with a higher PFC concentration would have more PFC pushed out of solution than a lower concentration solution for any given volume of water added.

Water concentrations between 30 and 60 vol % generally resulted in the largest nucleated droplets, while the smallest droplets were observed at the extremes of the lowest and highest water and ethanol concentrations. The increase in droplet diameter with increasing water concentration (i.e., lower ethanol concentration) is expected. As more water was introduced into the sample, the solvent phase became a poorer solvent for dissolved PFC. As a result, a greater concentration of PFC lost solubility with increasing water concentration, producing larger droplets. However, if the water concentration was sufficiently high (i.e., ethanol concentration <35%), the droplet diameter again started to decrease with increasing water concentration. At the lowest ethanol concentrations, droplet nucleation was likely limited by diffusion due to the low PFC concentrations present. This limited the concentration of PFC molecules within a finite diffusion radius of growing drop nuclei, producing smaller droplets at lower ethanol (i.e., lower PFC/higher water) concentrations.

Without wishing to be bound by theory, it is believed that when the ethanol concentration was low, overall oil concentration was also low. At the opposite extreme, when the ethanol concentration was high, it is plausible that only a low amount of PFC was forced out of solution whereas the majority of PFC was still dissolved in the continuous phase. Thus, at both extremes a low volume of excess PFC oil is forced out of solution and small droplets are produced.

Although PFC concentrations in solution after synthesis were typically under 1 vol. %, the number concentration of droplets formed using the ouzo synthesis method was as high as 10¹² droplet/ml. The droplet concentrations were estimated based off of back calculations from the volume of PFC dissolved, the droplet size distribution measured, and assuming that all the PFC was pushed out of solution to form droplets. Gravimetric measurements showed little PFC loss is observed with the ouzo synthesis method, because little mechanical energy or heat is generated during droplet nucleation. In contrast, during emulsification via sonication or homogenization, PFC losses up to 80% occur because input energy can vaporize large amounts of volatile oils. Depending on droplet size and ethanol content used during synthesis, final droplet concentrations in this work varied from 10¹⁰ droplet/ml (for ˜1 μm diameter droplets) to up to 10¹² droplet/ml (for ˜100 nm diameter droplets).

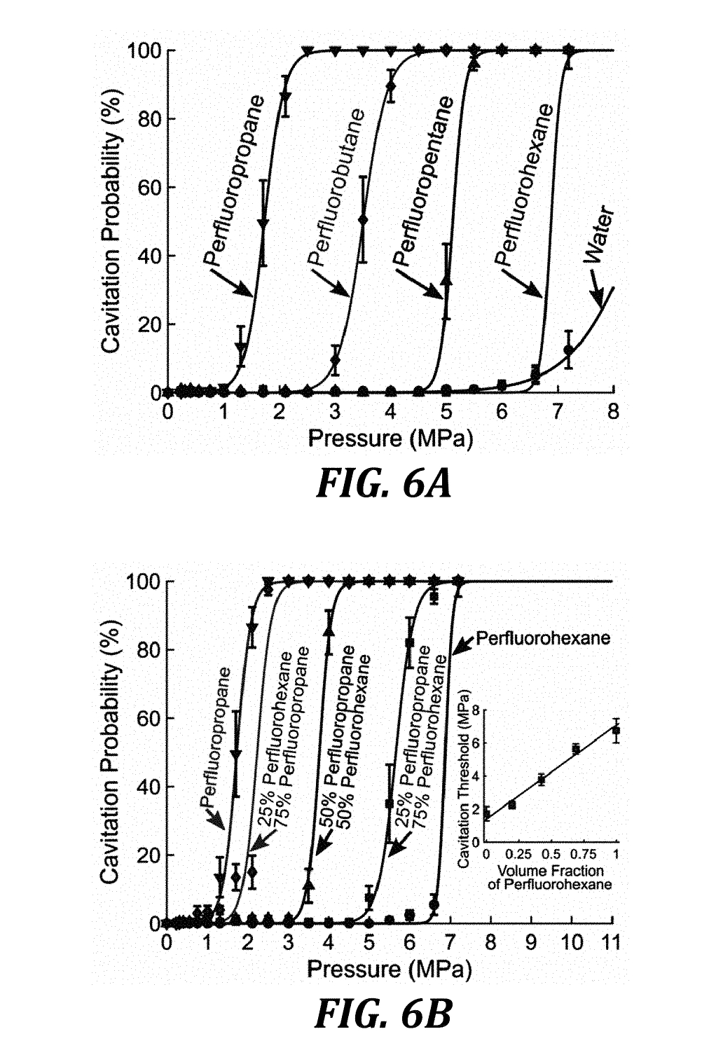

Activation thresholds for droplets synthesized with various PFC were also measured using an acoustic cavitation setup. The droplets were activated using short pulses (15 cycles) from a 1.24 MHz focused transducer in a degassed water bath held at body temperature (37° C.). The 50% activation (cavitation) threshold for perfluorohexane (T_{Boiling}=56° C.), perfluoropentane (T_{Boiling}=29° C.), perfluorobutane (T_{Boiling}=−2° C.), and perfluoropropane (T_{Boiling}=−37° C.) were 6.86 MPa, 5.11 MPa, 3.49 MPa, and 1.74 MPa (FIG. 6A), respectively. The pressure threshold to vaporize droplets was directly correlated with the boiling point of the PFC, as the boiling point is an indicator of PFC volatility.

The activation threshold of ouzo PFC droplets could also be modulated using PFC blends (FIG. 6B). This was demonstrated by combining a high boiling point PFC (perfluorohexane) with a low boiling point PFC (perfluoropropane). The blended droplets were prepared by saturating two ethanol solutions separately, each with a different perfluorocarbon, and combining the two ethanol-PFC solutions to the desired mixture ratio prior to adding the water mixture to nucleate droplets. As the perfluoropropane concentration increased, the activation threshold decreased. Fitting a linear function between the cavitation thresholds of perfluorobutane and pefluorohexane revealed that the activation threshold of the PFC blended droplets can be estimated based on a volume fraction weighted sum of the activation thresholds of the PFCs used (R²=0.97). Having the option to tune the activation threshold using PFC blends would be beneficial in scenarios where a specific activation threshold is needed to optimize droplet stability and activation sensitivity.

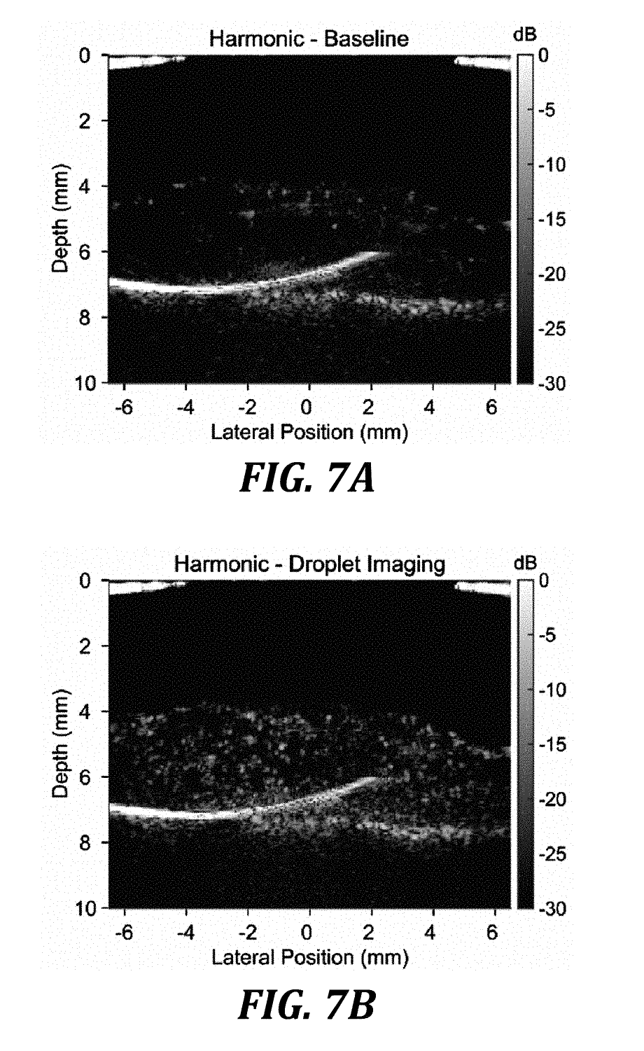

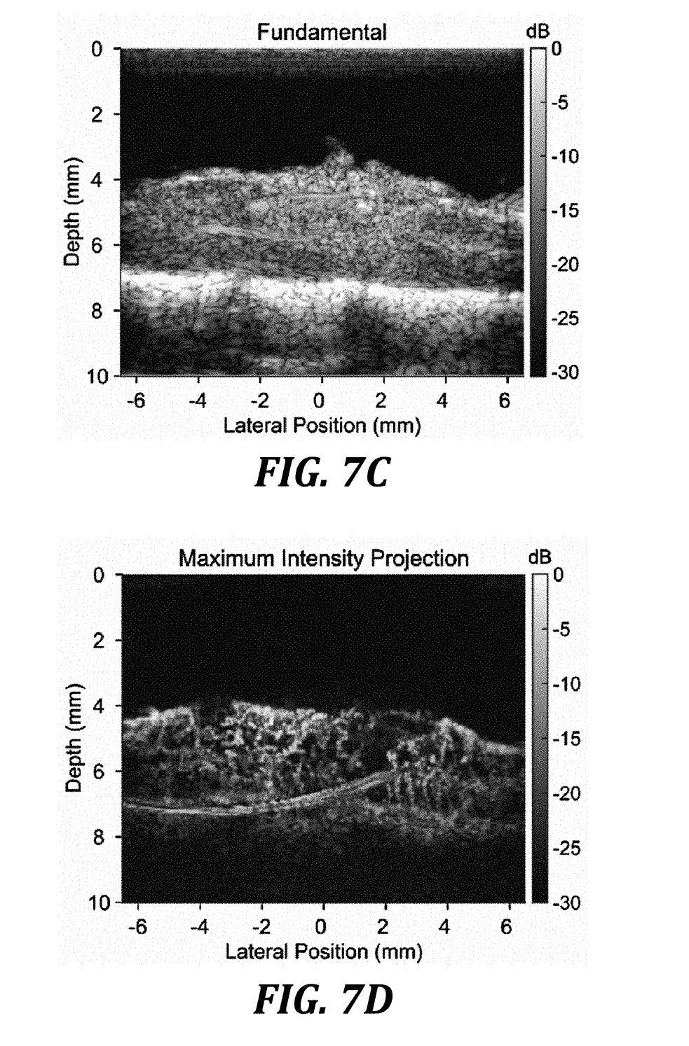

Ouzo-synthesized droplets were used as an ultrasound contrast agent in a rat spinal cord model (FIGS. 7A-7D). Surgical procedures were performed according to approved institutional animal care and use committee (IACUC) protocol following all appropriate guidelines from the university's Animal Welfare Assurance (A3464-01) as well as the NIH Office of Laboratory Animal Welfare (OLAW). A laminectomy was performed to remove the top surface of spine vertebrae, exposing the spinal cord. A bolus injection of perfluorobutane droplets (T_{Boiling}=29° C.) with a mean diameter of 182 nm was administered via tail vein and imaged using a 15 MHz linear array ultrasound transducer. Spinal cord tissue was seen at a depth between approximately 4 and 7 mm from the transducer face (FIG. 7C) on top of the vertebral bones of the spine. Although contrast from the droplets could not be easily seen in conventional ultrasound B-mode imaging (FIG. 7C), the agent was easily visualized using harmonic imaging (FIGS. 7B and 7C, see supplemental media). Harmonic imaging was necessary to suppress intrinsic linear signals from this tissue model and to highlight the non-linear signals that are generated from bubble oscillations. Detection of a harmonic signal after injection strongly suggests that droplets were successfully converted into nanobubbles or microbubbles. Maximum intensity projections (FIG. 7D) over a one second interval flowing bubble activation revealed the branched microvasculature of the spinal cord. Such images are examples of contrast-enhanced ultrasound (CEUS) images revealing details of tissue microcirculation.

At the frequency used, a low boiling point PFC droplet with a low activation threshold was needed for in vivo experiments because the pressure output from the high-frequency transducer was relatively low. Injections of higher boiling point PFCs, such as perfluoropentane (T_{Boiling}=29° C.) and perfluorohexane (T_{Boiling}=56° C.), provided nearly zero contrast enhancement since they were not activated by the clinical imaging transducer.

Based on these results, high volatility PFCs such as perfluorobutane (T_{Boiling}=−2° C.) and perfluoropropane (T_{Boiling}=−37° C.) are well suited for contrast-enhanced imaging using existing clinical imaging ultrasound due to their low acoustic activation thresholds. Having a low acoustic activation threshold would enable deeper droplet activation contrast enhanced imaging without exceeding FDA thresholds imposed on acoustic pressure. To develop the pressures needed to vaporize droplets several centimeters into tissue, a lower frequency transducer in the range of 4-8 MHz can be used instead of a 15 MHz transducer. By shifting to lower frequency transducers, acoustic attenuation would be reduced, and higher driving voltages could be achieved, resulting in greater acoustic pressures at depth. Moreover, it is possible that by using a lower frequency transducer the increased acoustic pressures at depth may be enough to vaporize higher boiling point perfluoropentane and perfluorohexane based droplets. Without wishing to be bound by theory, it is believed that these agents can quickly recondense back into their liquid phase after vaporization. Reversible vaporization and condensation has been shown to be beneficial in extending the lifetime of the agents after repeated activation cycles.

Therefore, in this Example, a new method to produce perfluorocarbon droplets using spontaneous nucleation has been developed and presented. The ouzo method is a fast and easy approach to produce nanodroplets with minimal equipment requirements and low costs. Although it was demonstrated here using fully fluorinated PFCs with boiling points ranging from −37° C. up to 56° C., the method can be extended to other types of PFCs or oils. Even though lipids were used in this study as a coating material, any alcohol or water-soluble surfactant material can be used. Methods for polymerizing shells on the droplet interface and/or the addition of targeting peptides can be easily adapted and incorporated into the ouzo method to produce PFC droplet based contrast agents for applications requiring agents with diameters less than 200 nm. Even though a large volume of ethanol is used to synthesize the droplets, excess ethanol is easily removed by dialysis or by centrifuging the droplets, decanting the solvent, and resuspension in fresh media. Because the ouzo method can consistently produce phase-change contrast agents with a diameter under 200 nm, the synthesis method can be used for applications in extravascular imaging and ultrasound-based therapies.

Droplet Synthesis:

Ouzo droplets were synthesized using both liquid and gaseous PFCs. The PFCs used included perfluorohexane (C₆F₁₄, T_{Boiling}=56° C., PFH), perfluoropentane (C₅F₁₂, T_{Boiling}=29° C., PFP), perfluorobutane (C₄F₁₀, T_{Boiling}=−2° C., PFB), and perfluoropropane (C₃F₈, T_{Boiling}=−37° C., OFP). All PFCs were purchased from SynQuest Laboratories. An initial lipid dissolved in ethanol stock solution was prepared using a 20:1 molar ratio of dipalmitoylphosphatidylcholine (DPPC, CAS: 63-89-8, NOF America Corp.) and N-(Methylpolyoxyethylene oxycarbonyl)-1,2-distearoyl-sn-glycero-3-phosphoethanolamine sodium salt (DSPE-PEG 2K, NOF America Corp.). The lipid concentration varied from 0.10 mg/ml up to 1.82 mg/ml depending on the volume of perfluorocarbon used in the synthesis.

The lipid-ethanol stock solution was then divided as needed. Using a stir plate, PFCs were then dissolved in the lipid-ethanol solution until it was fully saturated. For liquid PFCs (i.e. PFH and PFP), the PFC oil was incrementally added to the stirred ethanol solution until a small droplet of PFC formed in solution. The solution was then removed from the stir plate and allowed to rest at room temperature for approximately 20 minutes, enabling excess PFC to fall to the bottom of the container. For gaseous PFCs (i.e. PFB and OFP), PFC gas was bubbled into a sealed glass vial with the ethanol solution at a pressure of 1.5-2.0 PSI while on ice. It was bubbled through for a minimum of 2 minutes and purged 3 times to ensure the headspace was filled with gaseous PFC. Then, the PFC-lipid-ethanol solution was pipetted into a clean glass vial and diluted as needed using additional lipid-ethanol solution to achieve the desired PFC in ethanol saturation percentage. Finally, a 7:2:1 volume ratio blend of water, propylene glycol (CAS: 57-55-6, Sigma Aldrich), and glycerol (CAS: 56-81-5, Bio-Rad) was added to the solution to nucleate PFC droplets. Prior to measurements, the droplet samples were centrifuged, decanted, and resuspended to separate the droplets from excess alcohol and lipids. This process was repeated 3 times.

Droplet Size and Zeta Potential Measurements:

Droplet size distributions were measured using dynamic light scattering (DLS, Zetasizer NanoZS, Malvern Instruments Ltd., Worcestershire, UK) at 20° C. All samples were allowed to equilibrate in the sample holder for one to five minutes prior to measurement. Viscosity differences in solvent mixtures were corrected for by directly measuring the solvent mixture viscosity using a rheometer (Physica MCR301, Anton Paar, Graz, Austria) in a double gap cylinder (Couette) configuration. Droplet concentrations were estimated based on the volume of PFC introduced into the sample divided by the average diameter of the droplets nucleated.

Zeta potential measurements (Zetasizer NanoZS, Malvern Instruments Ltd., Worcestershire, UK) were all taken at 20° C. Droplet samples with a lipid shell were centrifuged and resuspended three times to remove any possible excess lipids in solution.

Acoustic Activation Threshold Measurements:

A passive cavitation detection method was used to detect droplet activation thresholds. Samples were held in a custom built thin-walled plastic cuvette (1 cm diameter, 4 cm long) submerged in a degassed water tank heated to body temperature (37° C.). Each sample was diluted to a concentration of approximately 10⁸ droplet/ml using degassed 0.45 μm filtered deionized water. To prevent droplet depletion due to vaporization, the sample holder was periodically flushed with deionized water and refilled with a new sample from the same batch. Depending on the acoustic pressure, samples were exchanged after a minimum of 200 and maximum of 1000 acoustic firings.

Droplets were activated using a 1.24 MHz spherically focused ultrasound transducer (H-102, f-number=0.95, D=68 mm, Sonic-Concepts Inc., Woodinville, Wash., USA). It was driven using a 15-cycle sine wave pulse generated from an arbitrary function generator (AFG 3022, Tektronix, Beaverton, Oreg., USA) and amplified by 55 dB using an RF amplifier (A-150, ENI, E&I Ltd., Rochester, N.Y., USA). Ultrasound pulses were delivered to samples with peak negative pressures ranging from 0 to 7.2 MPa at a pulse repetition frequency of 20 Hz.

Cavitation (activation) signals were detected using a custom-built, unfocused polyvinylidene difluoride (PVDF) transducer with near constant bandwidth up to 40 MHz. The PVDF transducer was positioned 35 mm away from the center of the sample holder orthogonal to the transmitting ultrasound transducer. Signals from this transducer were digitized and collected using a Gage card (Razor 14, Dynamic Systems LLC, Lockport, Ill., USA).

For the cavitation analysis, a 15 μs window, offset by the expected time delay for a one-way time of flight from the focused transducer to the sample then to the PVDF transducer, was used to analyze received ultrasound signals. The cavitation signal was identified by subtracting an averaged background acoustic signal from the signal acquired. A minimum of 200 acoustic signals were used for each acoustic condition for each sample. A cavitation event was identified as an average acoustic intensity 9 times greater than the background noise level. The cavitation (activation) probability was defined as the percentage of cavitation events registered versus the total number of acoustic pulses fired for a given acoustic condition. The activation threshold was defined as the 50% crossing found on a sigmoid fit of the cavitation probability versus pressure data collected.

In Vivo Imaging:

Surgical procedures were performed according to approved institutional animal care and use committee (IACUC) protocol following all appropriate guidelines from the university's Animal Welfare Assurance (A3464-01) as well as the NIH Office of Laboratory Animal Welfare (OLAW). A 0.2 ml bolus injection of perfluorobutane droplets diluted to approximately 10⁶ droplet/ml was injected via tail vein in an anesthetized female Sprague-Dawley rat. An ultrasound imaging window was made by performing a laminectomy to expose the spinal cord between T6 and T10. Activated droplets were imaged using a Verasonics Vantage ultrasound system (Verasonics Inc, Bothell, Wash., USA) and a 15 MHz transducer (Vermon, Tours, France). The spinal cord was imaged with conventional B-mode imaging and a plane-wave harmonic sequence used to differentiate the activated droplets from the surrounding tissue.

### Example 2. Lipid- or Albumin-Stabilized Nanoemulsions

A similar method to the method of Example 1 was used to synthesize lipid and albumin-stabilized PFC droplets. However, the lipids were dissolved in lipids at a concentration of 2.4 mM. The perfluorocarbon was dissolved in ethanol at a saturation of 20%. A 7:2:1 mixture of water:propylene glycol:glycerol was mixed to reach a final ethanol concentration of 20% by volume.

For albumin-stabilized droplets, water-soluble albumin was dissolved in water at a concentration of 4 mg/ml. The perfluorocarbon was dissolved in ethanol at a saturation of 20%. The water with albumin was mixed to reach a final ethanol concentration of 20% by volume.

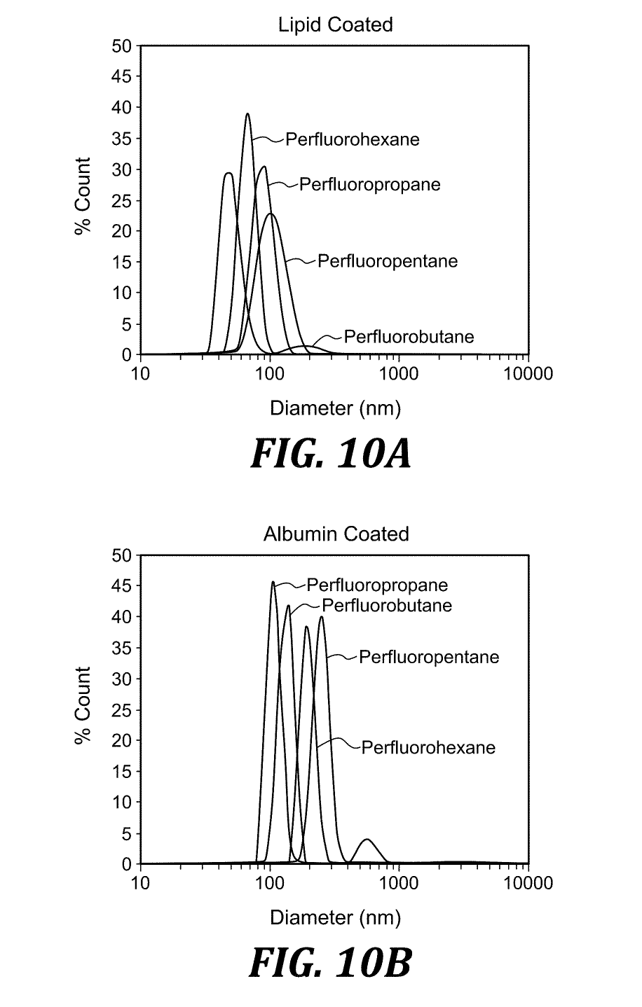

FIGS. 10A and 10B are dynamic light scattering measurements of perfluorocarbon nanodroplets produced with a (A) lipid and (B) albumin stabilizer. Lipid based stabilizers result in overall smaller diameter agents than albumin based stabilizers, which would be beneficial in applications where the agents are required to diffuse past vessel walls and into tissue. The two types of stabilizer coating used to stabilize these droplets are examples of FDA approved stabilizer coatings used in ultrasound nanobubble or microbubble contrast agents.

While illustrative embodiments have been illustrated and described, it will be appreciated that various changes can be made therein without departing from the spirit and scope of the disclosure.

The embodiments of the disclosure in which an exclusive property or privilege is claimed are defined as follows:

1. A kit, comprising:
a first compartment comprising a volatile fluorinated compound dissolved in a C₁₋₆ alcohol; and
a second compartment comprising water;
wherein the volatile fluorinated compound is present in the first compartment at a concentration of 0.2% or more and 4% or less by volume, and the volume ratio of the volatile fluorinated compound and C₁₋₆ alcohol:water is from 24:1 to 500:1.

2. The kit of claim 1, wherein the first and the second compartment are not in fluid communication with one another.

3. The kit of claim 2, wherein the first and the second compartments are separated by a frangible impermeable barrier.

4. The kit of claim 1, wherein the first and the second compartments are each a free-standing container.

5. The kit of claim 1, wherein the first compartment, the second compartment, or both the first and second compartments, further comprise a stabilizer.

6. The kit of claim 5, wherein the stabilizer is selected from a lipid, a protein, a polymer, and any combination thereof.

7. The kit of claim 1, wherein the first compartment, the second compartment, or both the first and second compartments further comprise a therapeutic agent.

8. The kit of claim 1, wherein the first compartment, the second compartment, or both the first and second compartments, further comprises a cell-targeting agent.

9. The kit of claim 1, wherein the first compartment, the second compartment, or both the first and second compartments, further comprise a diagnostic agent.

10. The kit of claim 1, wherein the C₁₋₆ alcohol is methanol or ethanol.

11. The kit of claim 1, wherein the volatile fluorinated compound is selected from sulfur hexafluoride, perfluorohexane, perfluoropentane, perfluorobutane, perfluoropropane, and any combination thereof.

12. The kit of claim 1, wherein the second compartment further comprises a co-solvent, a salt, a buffering agent, a sugar, or any combination thereof.

13. A nanodroplet composition, comprising:
a dispersion of nanodroplets of a volatile fluorinated compound in an aqueous liquid, wherein the nanodroplets have an average volume of 1.1×10⁵ nm³ or more and 4.2×10⁹ nm³ or less; and
the nanodroplets are present in the aqueous liquid at a concentration ranging from 1×10⁷ to 1×10¹⁴ nanodroplets per ml of aqueous liquid.

14. The nanodroplet composition of claim 13, wherein the nanodroplets are in a liquid phase and the volatile fluorinated compound is selected from sulfur hexafluoride, perfluorohexane, perfluoropentane, perfluorobutane, perfluoropropane, and any combination thereof.

15. The nanodroplet composition of claim 13, wherein the composition further comprises a buffering agent and a C₁₋₆ alcohol.

16. The nanodroplet composition of claim 13, wherein the composition further comprises a therapeutic agent, a targeting agent, a diagnostic agent, or any combination thereof.

17. A method for making the nanodroplet composition, comprising:
providing a first solution comprising a volatile fluorinated compound dissolved in a C₁₋₆ alcohol and a second solution comprising water; and
mixing the first and second solutions to provide the nanodroplet composition of claim 13.

18. A method of generating nanobubbles or microbubbles, comprising:
exposing the nanodroplet composition of claim 13 to ultrasound to vaporize the nanodroplets, thereby providing nanobubbles or microbubbles having a diameter of ranging from 120 nm to 10 μm.

19. A therapeutic or diagnostic method, comprising:
administering a nanodroplet composition to a subject in need thereof, wherein the nanodroplet composition comprises a dispersion of nanodroplets of a volatile fluorinated compound in an aqueous liquid, wherein the nanodroplets have an average volume of 1.1×10⁵ nm³ or more and 4.2×10⁹ nm³ or less, and the nanodroplets are present in the aqueous liquid at a concentration of from 1×10⁷ to 1×10¹⁴ nanodroplets per ml of aqueous liquid; and
exposing the injected nanodroplet composition to ultrasound to vaporize the nanodroplets, thereby providing nanobubbles or microbubbles having a diameter of from 120 nm to 10 μm.

20. The therapeutic or diagnostic method of claim 19, further comprising, prior to injecting the nanodroplet composition, mixing a first solution comprising a volatile fluorinated compound in a C1-6 alcohol and a second solution comprising water to provide the nanodroplet composition.
